(19) United States
(12) Patent Application Publication
(10) Pub. No.: US 2017/0042704 A1
(43) Pub. Date: Feb. 16, 2017

(54) EXOSKELETON-TYPE GLOVE

(71) Applicant: KOREA UNIVERSITY OF TECHNOLOGY AND EDUCATION INDUSTRY-UNIVERSITY COOPERATION FOUNDATION, Cheonan-si (KR)

(72) Inventors: Jee-Hwan RYU, Cheonan-si (KR); Dmitry POPOV, Cheonan-si (KR); Igor GAPONOV, Cheonan-si (KR)

(73) Assignee: KOREA UNIVERSITY OF TECHNOLOGY AND EDUCATION INDUSTRY-UNIVERSITY COOPERATION, Cheonan-si (KR)

(21) Appl. No.: 15/305,195

(22) PCT Filed: Feb. 10, 2015
(86) PCT No.: PCT/KR2015/001349

§ 371 Date: Oct. 19, 2016

(30) Foreign Application Priority Data
Apr. 21, 2014 (KR) 10-2014-0047473
Apr. 21, 2014 (KR) 10-2014-0047474

(51) Int. Cl. A61F 2/68; A61F 2/58

(52) U.S. Cl. CPC A61F 2/68; A61F 2/583; A61F 2002/701

(57) ABSTRACT

Provided is an exoskeleton-type glove including: a glove type outer cover; a guide provided at a corresponding position between finger joints of a human body on the outer cover; a wire extended along the guide; an actuation module including an actuator that allows the finger joint of the human body to move by controlling an extension length of the wire and provided at a back side of a hand of the human body; and a buffer provided below the actuation module.

### TECHNICAL FIELD

The present invention relates to an exoskeleton-type glove.

### BACKGROUND ART

Multiple auxiliary apparatuses of a wearing or mounting type have been developed in order to assist body functions of persons which do not have a part of a body or cannot move a part of the body or in order to improve a function of the body itself In particular, an auxiliary apparatus has been developed, which a person wears by being worn on a hand in a glove form in order to assist a motion of a finger.

The glove form auxiliary apparatus in the related art includes an actuator that moves a joint for each joint of each finger by using an exoskeleton structure and generally includes a sensor that senses a variation or an angle at which the joint moves.

However, since the auxiliary apparatus controls the joint by mechanical force, there are a lot of cases in which a user feels pain by a pressure generated during actuation. In particular, since a degree of the pain further increases as larger actuation force is generated, it is actually difficult to generate force as large as the user wants.

Therefore, a method for solving the problems is requested.

### DISCLOSURE

### Technical Problem

An object of the present invention is to provide an exoskeleton-type glove which can simplify an apparatus and reduce a total volume and a total weight of the apparatus.

Another object of the present invention is to provide an exoskeleton-type glove which can significantly alleviate pain felt by a user during actuation.

Yet another object of the present invention is to provide an exoskeleton-type glove which can automatically start or terminate a grasp operation by sensing a grasp motion of a finger of a human body.

The objects of the present invention are not limited to the aforementioned objects, and other objects, which are not mentioned above, will be apparent to a person having ordinary skill in the art from the following description.

### Technical Solution

In order to achieve the object, an exoskeleton-type glove according to the present invention includes: a glove type outer cover; a guide provided at a corresponding position between finger joints of a human body on the outer cover; a wire extended along the guide; an actuation module including an actuator that allows the finger joint of the human body to move by controlling an extension length of the wire and provided at a back side of a hand of the human body; and a buffer provided below the actuation module.

In addition, the actuation module may further include a fixation case fixing the actuator at the back side of the hand of the human body.

Further, the fixation case may include a base part having a seating surface seated on a back of the hand of the human body, and a pair of fixation parts extended upwards at both ends of the base part and fixing the actuator.

Moreover, the fixation case may further include a pulley formed at at least any one side of the one pair of fixation parts and rotatably provided by the actuator to wind or unwind the wire.

Besides, the fixation case may further include a separation preventing member formed to cover at least a partial circumference of the pulley to prevent the wire from being separated.

In addition, the exoskeleton-type glove may further include an auxiliary buffer mounted on a hand of the human body.

Further, the exoskeleton-type glove may further include a plurality of sensors sensing grasp motions of the finger of the human body, wherein the actuation module may allow the finger joint of the human body to move by controlling an extension length of the wire according to sensing results of the plurality of sensors.

In addition, the grasp motions of the human body may include an open motion and a close motion.

Moreover, the plurality of sensors may include a sensor provided facing a lower part of the end of the finger of the human body in the outer cover to sense a start of the close motion among the grasp motions of the finger of the human body.

In addition, the plurality of sensors includes a sensor provided at a position corresponding to the lower part of the end of the finger of the human body on the outer cover to sense a termination of the close motion among the grasp motions of the finger of the human body.

Moreover, the plurality of sensors may include a sensor provided facing an upper part of the end of the finger of the human body in the outer cover to sense the start of the open motion among the grasp motions of the finger of the human body.

In addition, the exoskeleton-type glove may further include a fixation plate disposed on the outer cover to be separated from the surface of the outer cover, wherein the plurality of sensors may include a sensor provided below the fixation plate by facing a first knuckle of the human body to sense the termination of the open motion among the grasp motions of the finger of the human body.

Moreover, the plurality of sensors may be a button type sensor.

### Advantageous Effects

The exoskeleton-type glove of the present invention achieves the following effects.

First, an unnecessary apparatus is omitted and as an actuation module is provided at a back of a hand of a human body, each component is rationally disposed, and as a result, an entire apparatus is simplified and a volume and a weight are reduced.

Second, pain felt by a user during actuation can be significantly alleviated.

Third, a motion of a finger joint of the user can be effectively assisted.

The effects of the present invention are not limited to the aforementioned effect, and other effects, which are not mentioned above, will be apparent to a person having ordinary skill in the art from description of claims.

### DESCRIPTION OF DRAWINGS

FIG. 1 is a plan view illustrating a whole shape of an exoskeleton-type glove according to an embodiment of the present invention;

FIG. 2 is a plan view conceptually illustrating a connection shape of respective components in the exoskeleton-type glove according to the embodiment of the present invention;

FIG. 3 is a bottom view conceptually illustrating the connection shape of the respective components in the exoskeleton-type glove according to the embodiment of the present invention;

FIG. 4 is a perspective view illustrating a shape of a fixation case in the exoskeleton-type glove according to the embodiment of the present invention;

FIG. 5 is a perspective view illustrating a shape of a pulley provided in the fixation case in the exoskeleton-type glove according to the embodiment of the present invention;

FIG. 6 is a side view illustrating a shape in which a buffer is provided between an actuation module and a back of a hand of a human body in the exoskeleton-type glove according to the embodiment of the present invention;

FIG. 7 is a side view illustrating a shape in which a predetermined object is gripped by actuating the exoskeleton-type glove according to the embodiment of the present invention;

FIG. 8 is a side view illustrating a shape in which a plurality of sensors is provided in the exoskeleton-type glove according to the embodiment of the present invention;

FIG. 9 is a side view illustrating a shape in which a closing operation is performed during a grasp operation by actuating the exoskeleton-type glove according to the embodiment of the present invention; and

FIG. 10 is a side view illustrating a shape in which an operating operation is performed during the grasp operation by actuating the exoskeleton-type glove according to the embodiment of the present invention.

### BEST MODE

Hereinafter, a preferred embodiment of the present invention, in which a purpose of the present invention can be realized in detail will be described with reference to the accompanying drawings. In describing the embodiment, the same name and the same reference numerals are used with respect to the same component and the resulting additional description will be omitted.

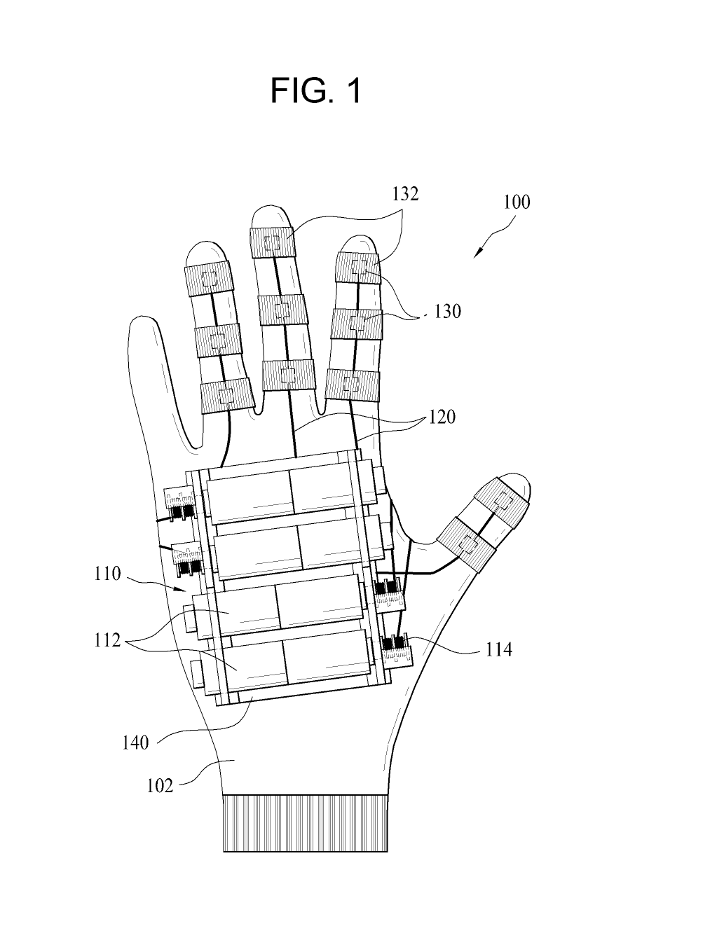

FIG. 1 is a plan view illustrating a whole shape of an exoskeleton-type glove according to an embodiment of the present invention.

As illustrated in FIG. 1, the exoskeleton-type glove 100 according to the embodiment of the present invention includes a guide 130, a wire 120 and an actuation module 110.

The guide 130 may be provided at a corresponding position between finger joints of a human body and provided even at other portions other than the finger joints. In addition, the guide 130 is provided at each predetermined position of the wire 120 to fix the wire 120 and enable the wire 120 to slidably move.

That is, the wire 120 may extend along a predetermined path by the guide 130. In the case of the embodiment, although not illustrated, the guide 130 has a through-hole through which the wire 120 passes to prevent the wire 120 from being separated and enable the wire 120 to move in a longitudinal direction. Even in embodiments other than the embodiment, a shape, a structure, and an installation position, and the like of the guide 130 may be variously formed, of course.

Further, in the embodiment, a protection cover 132 is provided at each joint so as to prevent the guide 130 provided at each finger joint from being exposed to the outside.

The actuation module 110 includes an actuator 112 that controls the finger joint of the human body to move by controlling the wire 120 and is provided at a back of a hand of the human body. In the exoskeleton-type glove 100 of the present invention, since the actuation module 110 is positioned at the back of the hand, respective components of the actuation module 110 are not distributed to further simplify an apparatus and prevent a motion of the finger joint from being interfered by the actuation module 110.

In this case, the actuator 112 may be formed in various types including a rotary motor, and the like and in the embodiment, the actuator 112 is formed to control an extension length of the wire 120 by winding or unwinding the wire 120 with rotation. That is, the actuation module 110 may control the finger joint of the human body by controlling the extension length of the wire 120. In addition, a pulley 114 of the fixation case 140 is connected to one side of the actuator 112 so as to wind the wire 120 and this will be described below.

Further, a plurality of actuators 112 may be provided according to the number of the wires 120. In the embodiment, a total of 8 wires 120 are formed to correspond to four finger outsides and insides of 4 fingerers among fingers of the human body, respectively and a total of 4 actuators 112 are provided to simultaneously control the wires 120 corresponding to the outsides and the insides of each finger.

However, the number of the wires 120 and the number of the actuators 112 may be variously changed according to a design, of course.

Meanwhile, in the case of the embodiment, the actuation module 110 further includes the fixation case 140 that fixes the actuator 112 at the back of the hand. The fixation case 140 may allow the actuator 112 to be easily fixed and this will be described below. Further, in the embodiment, the actuation module 110, the wire 120, and the guide 130 may be provided on a glove-type outer cover 102. That is, the outer cover 102 provides a glove type exterior, and as a result, the outer cover 102 enables a user to easily wear the exoskeleton-type glove and stably fixes each component.

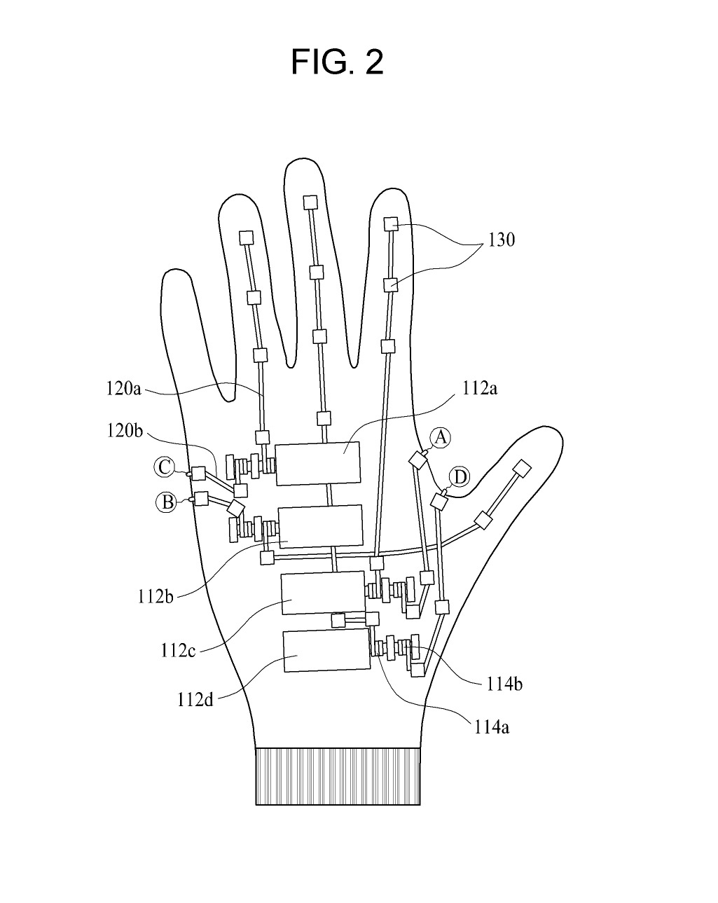

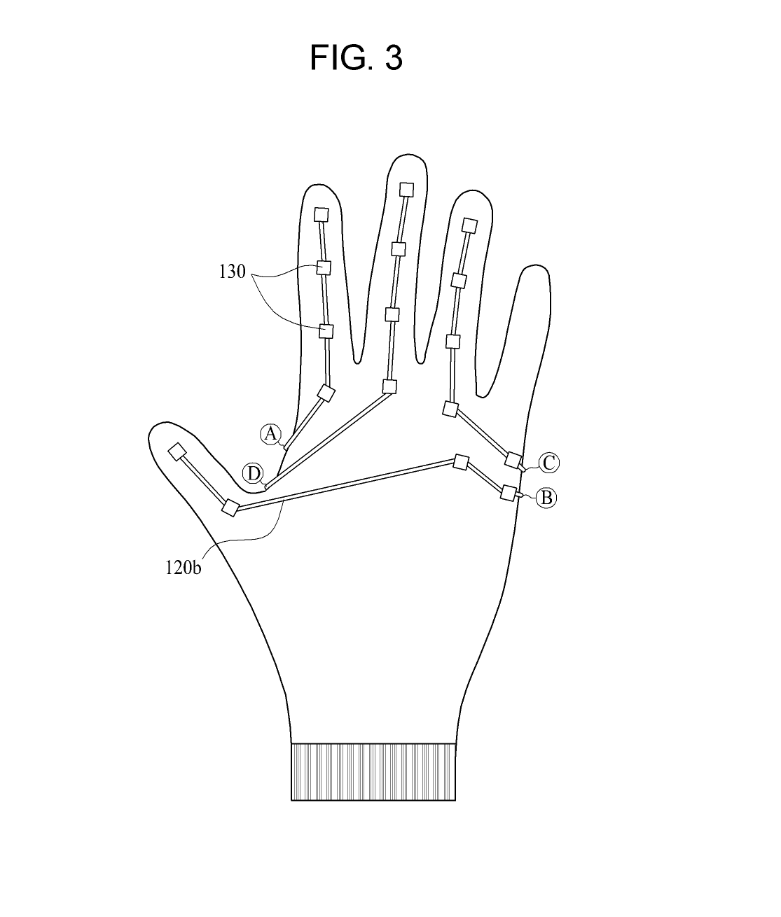

FIG. 2 is a plan view conceptually illustrating a connection shape of respective components in the exoskeleton-type glove 100 according to the embodiment of the present invention and FIG. 3 is a bottom view conceptually illustrating the connection shape of the respective components in the exoskeleton-type glove 100 according to the embodiment of the present invention.

Referring to FIGS. 2 and 3, the actuation module 110 includes a total of 4 actuators 112 in the embodiment as described above. In addition, a pair of wires 120 are formed to correspond to one pair of actuators 112 provided at the inside and the outside of one finger, respectively.

In the embodiment, in detail, a first actuator 112a corresponds to a ring finger, a second actuator 112b corresponds to a thumb, a third actuator 112c corresponds to a forefinger, and a fourth actuator 112d corresponds to a middle finger. Further, the respective actuators are connected with a first wire 120a which extends to the outside of each finger and connected with a second wire 120b which extends to the inside of each finger.

In addition, when the first wire 120a is provided while being wound on a first pulley 114a and the second wire 120b is provided while being wound on a second pulley 114b, and as a result, the actuator 112 rotates in any one direction, the first wire 120a may be unwound and the second wire 120b may be wound. In such a case, a finger may be controlled to be retracted and when the actuator 112 rotates in a reverse direction, the finger may be controlled to be opened.

Meanwhile, in the case of the embodiment, since the actuator 112 is provided at the back of the hand, a plurality of second wires 120b forms a path to cover the side of a hand from the back of the hand to extend to a palm. In FIGS. 2 and 3, A, B, C, and D represent portions where the respective second wires 120b are connected. By such a configuration, the second wire 120b may be prevented from interfering with the motion of the finger.

The embodiment as just one example and the number of the actuators 112, a connection relationship between the actuator 112 and the wire 120, an extension path of each wire 120, and the like may be, of course, designed variously without a limit.

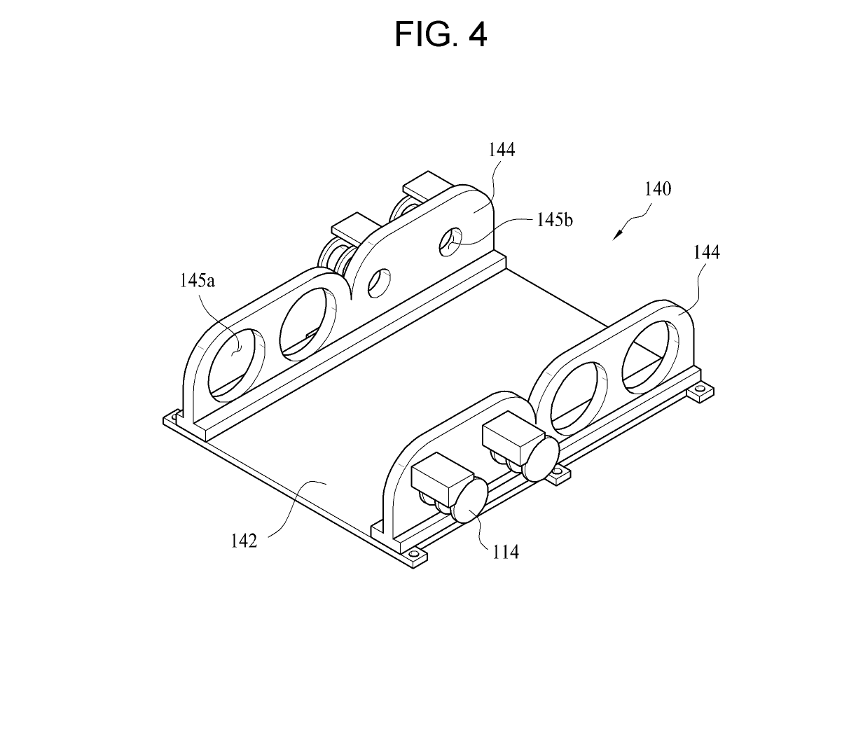

FIG. 4 is a perspective view illustrating a shape of a fixation case 140 in the exoskeleton-type glove 100 according to the embodiment of the present invention.

As illustrated in FIG. 4, in the embodiment, the actuation module 110 includes the fixation case 140. In addition, the fixation case 140 includes a base part 142 and a fixation part 144.

In detail, in the base part 142, a seating surface seated on the back of the hand of the human body is formed, and as a result, the base part 142 may contact the back of the hand throughout a wide area. When the area of the seating surface increases, a pressure applied to the back of the hand of the user may be more widely distributed.

A pair of fixation parts 144 are formed to extend upwards at both ends of the base part 142 and fix the actuator 112. In the case of the embodiment, the fixation part 144 has a first fixation hole 145a to which one side of a body of the actuator 112 is fixed and a second fixation hole 145b to which the other side of the body of the actuator 112 is fixed.

In addition, the fixation case 140 may further include the pulley 114 that is formed at at least any one side of the one pair of fixation part 144 and rotatably provided by the actuator 112 to wind or unwind the wire 120. In the embodiment, the pulley 114 is connected with a rotary shaft of the actuator 112 to rotate in one direction or the other direction by driving the actuator 112.

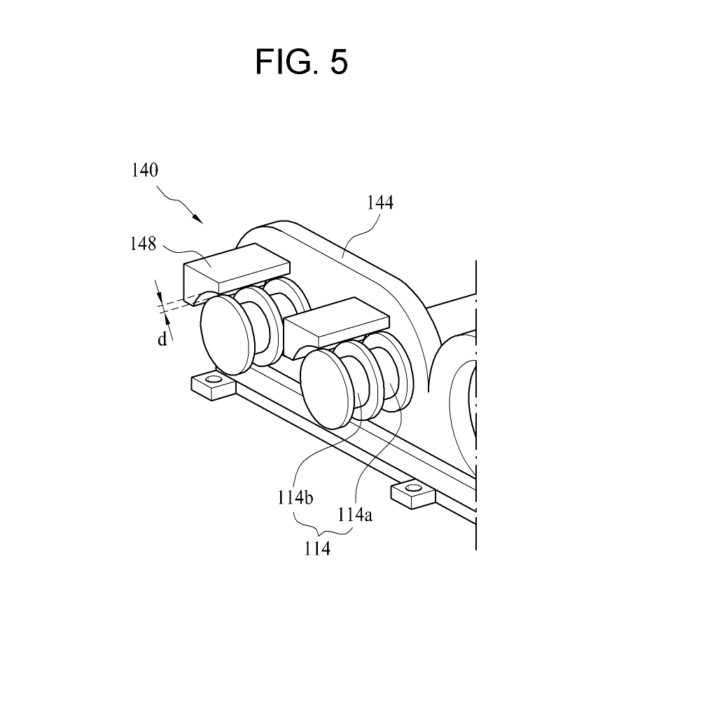

FIG. 5 is a perspective view illustrating a shape of a pulley 114 provided in the fixation case 140 in the exoskeleton-type glove 100 according to the embodiment of the present invention.

As illustrated in FIG. 5, the pulley 114 is constituted by a first pulley 114a winding or unwinding the first wire 120a and a second pulley 114b winding or unwinding the second wire 120b.

In this case, the fixation case 140 may further include a separation preventing member 148 formed to cover at least a partial circumference of the pulley 114 to prevent the wire 120 from being separated. That is, the separation preventing member 148 is installed adjacent to the pulley 114 in order to prevent the wire 120 wound on the pulley 114 from being separated from the pulley 114 while the wire 120 wound on the pulley 114 is wound or unwound by driving the actuation module 110.

In this case, a distance d with the circumference of the pulley 114 and the separation preventing member 148 are separated from each other may be formed to be smaller than the thickness of the wire 120.

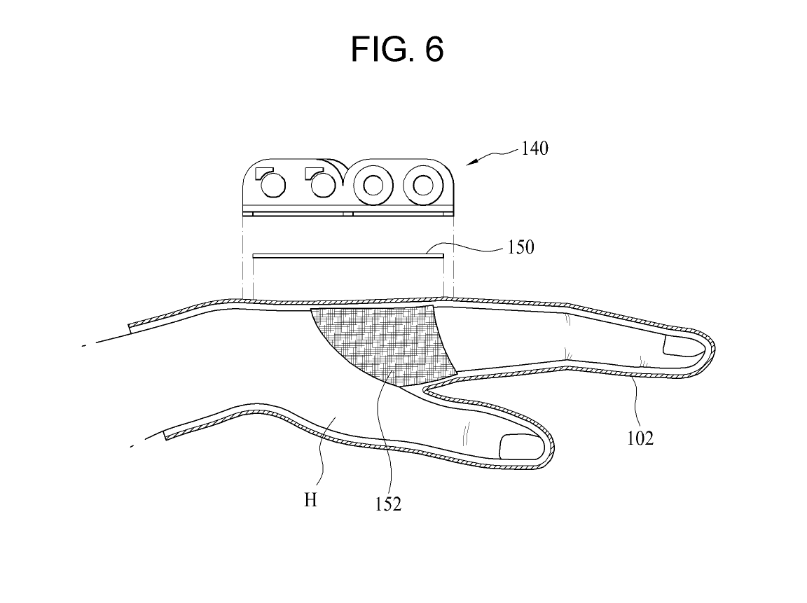

FIG. 6 is a side view illustrating a shape in which a buffer 150 is provided between an actuation module 110 and a back of a hand of a human body in the exoskeleton-type glove 100 according to the embodiment of the present invention.

As illustrated in FIG. 6, in the embodiment, the buffer 150 is further provided between the fixation case 140 of the actuation module 110 and the back of the hand of the human body. The buffer 150 alleviates the pain felt by the user due to the pressure generated in the course of controlling the joint by mechanical force and the buffer 150 may be formed to have elasticity.

In addition, an area of the buffer 150 may be at least larger than the base part 142 of the fixation case 140.

Meanwhile, the buffer 150 may be provided below the actuation module 110 including the fixation case 140 and below the wire 120. In this case, the buffer 150 is provided while being fitted between the wire 120 and the outer cover 102 to alleviate friction and the pain depending on movement of the wire 120.

Further, in the embodiment, an auxiliary buffer 152 directly mounted on a hand H of the user is further provided inside the outer cover 102. The auxiliary buffer 152 may further alleviate the pain felt by the user together with the buffer 150. Therefore, since the user does not almost feel the pain in spite of further increasing the actuation force of the actuation module 110, larger force may be generated.

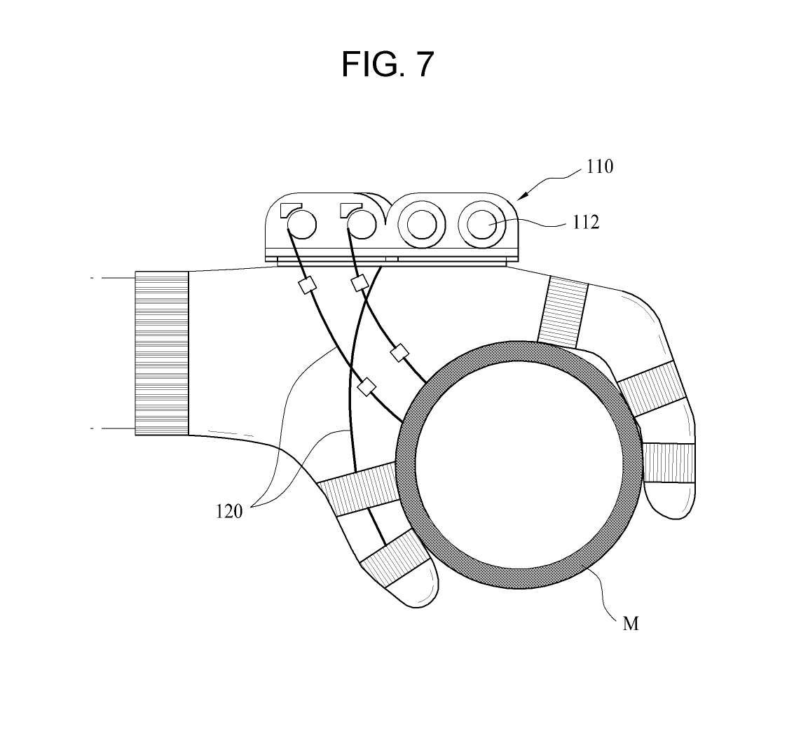

FIG. 7 is a side view illustrating a shape in which a predetermined object M is gripped by actuating the exoskeleton-type glove 100 according to the embodiment of the present invention.

As illustrated FIG. 7, the exoskeleton-type glove 100 according to the embodiment of the present invention may assist the user to grip the predetermined object M. This may be used for normal living even when the user loses a body function or may be easily used even in the case of gripping the object M having a weight which exceeds a muscular strength of the user.

That is, when the predetermined object M is gripped, the actuator 112 of the actuation module 110 rotates in one direction to control the finger to be retracted by winding the wire 120 at the palm side and unwinding the wire 120 at the back side of the hand to press the object M. Further, when the user intends to cancel the grip of the object M, the actuator 112 rotates in other direction to unwind the wire 120 at the palm side and wind the wire 120 at the back side of the hand, thereby controlling the finger to be stretched.

Meanwhile, in the embodiment, the exoskeleton-type glove 100 may be actuated in a manual mode and/or an automatic mode.

When the exoskeleton-type glove 100 is actuated in the manual mode, the user may actuate the exoskeleton-type glove 100 by operating a joystick (not illustrated). For example, operation buttons including an opening button and/or a closing button may be provided in the joystick.

When the exoskeleton-type glove 100 is actuated in the automatic mode, an object sensor (not illustrated) such as an infrared sensor, or the like is provided in a palm part of the outer cover 102 and when the predetermined object is sensed by the object sensor, the exoskeleton-type glove 100 may perform a grasp operation.

Alternatively, an electromyography (EMG) sensor is disposed at a forearm of the human body, and the like and senses electrical activity of a muscle of the forearm and the exoskeleton-type glove 100 may perform the grasp operation according to a sensing result. A flex sensor is disposed according to the finger of the human body, minute motions of some fingers are sensed, and the exoskeleton-type glove 100 will be able to perform the grasp operation according to the sensing result.

Meanwhile, the grasp operation of the exoskeleton-type glove 100 is divided into a plurality of operations and sensors for sensing the respective operations are disposed on the outer cover 102 to control the grasp operation of the exoskeleton-type glove 100. Hereinafter, the exoskeleton-type glove 100 to which the method is applied will be described.

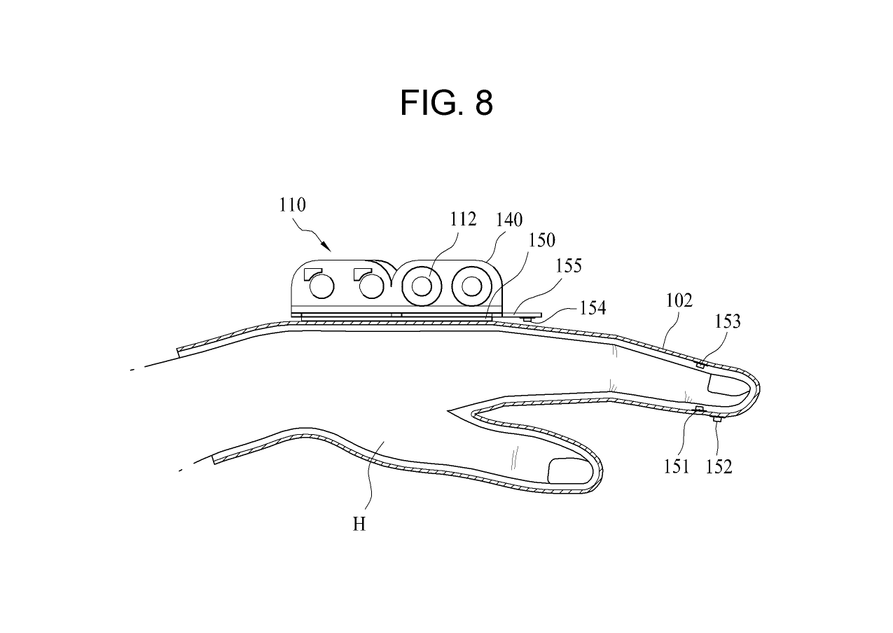

FIG. 8 is a side view illustrating a shape in which a plurality of sensors is provided in the exoskeleton-type glove according to the embodiment of the present invention.

Referring to FIG. 8, a plurality of sensors 151 to 154 sensing a grasp motion of the finger of the human body is provided. For example, the plurality of sensors 151 to 154 may be button type sensors, but the present invention is not limited thereto and various sensors that may sense a contact, a pressure, a motion, and the like may be adopted.

The grasp motion of the finger of the human body may include an open motion and a close motion. In detail, the grasp motion may be divided into start and termination of the open motion and the start and the termination of the close motion. The start and the termination of the open motion are associated with a motion of retracting the finger in order to grasp the object and the start and the termination of the close motion are associated with a motion of stretching the finger in order to cancel the grasp of the object.

The grasp operation of the exoskeleton-type glove 100 may also be performed to correspond to the grasp motion of the finger of the human body.

In addition, among the plurality of sensors, the sensor 151 provided facing a lower part of the end of the finger of the human body in the outer cover 102 may sense the start of the close motion among the grasp motions of the finger of the human body.

Further, the sensor 152 provided at a position corresponding to the lower part of the end of the finger of the human body on the outer cover 102 may sense the termination of the close motion among the grasp motions of the finger of the human body.

In addition, the sensor 153 provided facing an upper part of the end of the finger of the human body in the outer cover 102 may sense the start of the open motion among the grasp motions of the finger of the human body.

Meanwhile, a fixation plate 141 may be disposed to extend from the fixation case 140. The fixation plate 141 may be disposed to be separated from the surface of the outer cover 102 by a predetermined distance. The fixation plate 141 may be disposed at a position corresponding to the back of the hand of the human body. The fixation plate 141 may be formed apart from or integrally with the fixation case 140. In addition, the sensor 154 provided below the fixation plate 141 by facing a first knuckle of the finger of the human body may sense the termination of the open motion among the grasp motions of the finger of the human body.

The plurality of sensors 151 to 154 may be disposed at the forefinger among the fingers of the human body, but the present invention is not limited thereto. The plurality of sensors 151 to 154 may be disposed at all or some of 4 fingers other than the thumb among the fingers of the human body and variously changed according to the design.

The actuator 112 of the actuation module 110 may rotate in one direction or other direction according to sensing results of the plurality of sensors 151 to 154 or stop rotating.

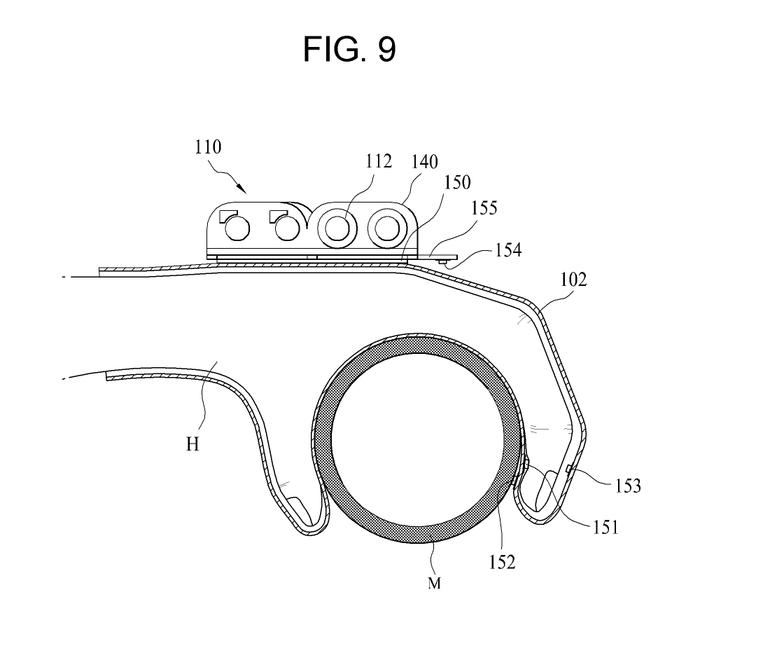

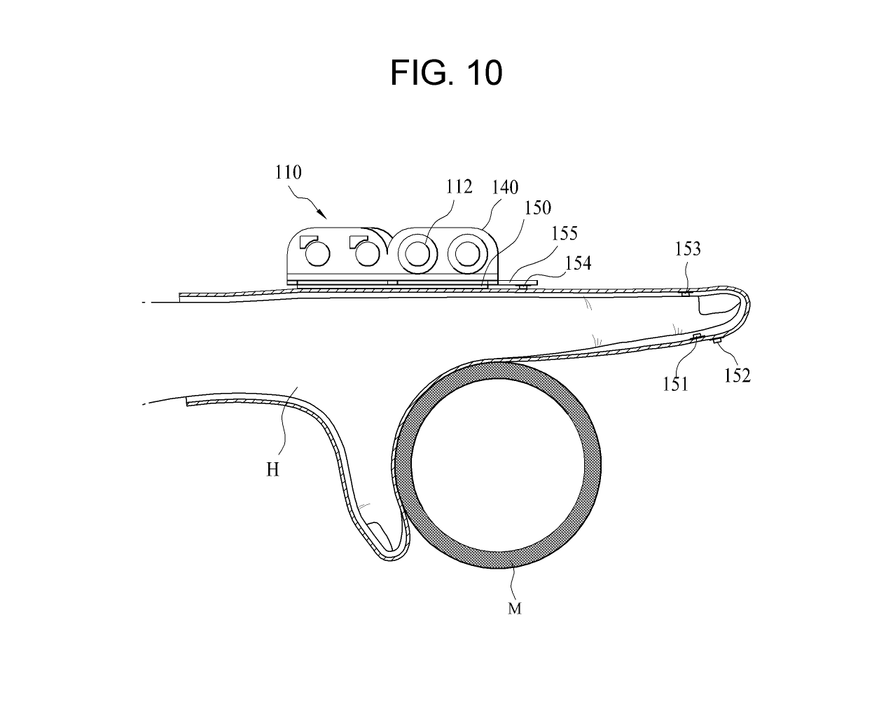

FIG. 9 is a side view illustrating a shape in which a closing operation is performed during a grasp operation by actuating the exoskeleton-type glove according to the embodiment of the present invention and FIG. 10 is a side view illustrating a shape in which an operating operation is performed during the grasp operation by actuating the exoskeleton-type glove according to the embodiment of the present invention.

First, as illustrated in FIG. 9, when the lower part of the end of the finger of the user presses the sensor 151, the sensor 151 may sense the pressing as the start of the close motion among the grasp motions. As a result, in order to start the close operation, the actuator 112 of the actuation module 110 rotates in one direction to control the finger of the user to be retracted by winding the wire 120 at the palm side and unwinding the wire 120 at the back side of the hand.

Thereafter, as the finger of the user is retracted, when the object M or another finger (that is, the thumb) of the user presses the sensor 152, the sensor 152 may sense the pressing as the termination of the close motion among the grasp motions. As a result, in order to terminate the close operation, the actuator 112 of the actuation module 110 stops rotating in one direction. That is, the actuator 112 of the actuation module 110 continuously rotates in one direction until the object M presses the sensor 152 when grasping the object M is completed.

Next referring to FIG. 10, when the upper part of the end of the finger of the user presses the sensor 153, the sensor 153 may sense the pressing as the start of the open motion among the grasp motions. As a result, in order to start the open operation, the actuator 112 of the actuation module 110 rotates in other direction to control the finger to be stretched by unwinding the wire 120 at the palm side and winding the wire 120 at the back side of the hand.

Thereafter, as the finger of the user is stretched, when the first knuckle of the finger of the user presses the sensor 154, the sensor 154 may sense the pressing as the termination of the open motion among the grasp motions. As a result, in order to terminate the open operation, the actuator 112 of the actuation module 110 stops rotating in other direction. That is, the actuator 112 of the actuation module 110 continuously rotates in other direction until canceling grasping of the object M is completed and the first knuckle of the finger thus presses the sensor 154.

As described above, the exoskeleton-type glove 100 according to the embodiment of the present invention may assist the user to grip the predetermined object M. This may be used for normal living even when a body function is lost or may be easily used even in the case of gripping the object M having a weight which exceeds a muscular strength of the user.

A preferred embodiment of the present invention has been described as above and a fact that the present invention can be materialized in other specific forms without departing from the gist or scope even except for the above described embodiment is apparent to those skilled in the art. Therefore, the aforementioned embodiment is not limited but needs to be exemplary, and as a result, the present invention is not limited to the above description and modified within the scope of the appended claims and a range equivalent thereto.

1. An exoskeleton-type glove comprising:
a glove type outer cover;
a guide provided at a corresponding position between finger joints of a human body on the outer cover;
a wire extended along the guide;
an actuation module including an actuator that allows the finger joint of the human body to move by controlling an extension length of the wire and provided at a back side of a hand of the human body; and
a buffer provided below the actuation module.

2. The exoskeleton-type glove of claim 1, wherein the actuation module further includes a fixation case fixing the actuator at the back side of the hand of the human body.

3. The exoskeleton-type glove of claim 2, wherein the fixation case includes
a base part having a seating surface seated on a back of the hand of the human body, and
a pair of fixation parts extended upwards at both ends of the base part and fixing the actuator.

4. The exoskeleton-type glove of claim 3, wherein the fixation case further includes a pulley formed at at least any one side of the one pair of fixation parts and rotatably provided by the actuator to wind or unwind the wire.

5. The exoskeleton-type glove of claim 4, wherein the fixation case further includes a separation preventing member formed to cover at least a partial circumference of the pulley to prevent the wire from being separated.

6. The exoskeleton-type glove of claim 1, further comprising:
an auxiliary buffer mounted on a hand of the human body.

7. The exoskeleton-type glove of claim 1, further comprising:
a plurality of sensors sensing grasp motions of the finger of the human body,
wherein the actuation module allows the finger joint of the human body to move by controlling an extension length of the wire according to sensing results of the plurality of sensors.

8. The exoskeleton-type glove of claim 7, wherein the grasp motions of the finger of the human body include an open motion and a close motion.

9. The exoskeleton-type glove of claim 8, wherein the plurality of sensors includes a sensor provided facing a lower part of the end of the finger of the human body in the outer cover to sense a start of the close motion among the grasp motions of the finger of the human body.

10. The exoskeleton-type glove of claim 8, wherein the plurality of sensors includes a sensor provided at a position corresponding to the lower part of the end of the finger of the human body on the outer cover to sense a termination of the close motion among the grasp motions of the finger of the human body.

11. The exoskeleton-type glove of claim 8, wherein the plurality of sensors includes a sensor provided facing an upper part of the end of the finger of the human body in the outer cover to sense the start of the open motion among the grasp motions of the finger of the human body.

12. The exoskeleton-type glove of claim 8, further comprising:
a fixation plate disposed on the outer cover to be separated from the surface of the outer cover,
wherein the plurality of sensors includes a sensor provided below the fixation plate by facing a first knuckle of the human body to sense the termination of the open motion among the grasp motions of the finger of the human body.

13. The exoskeleton-type glove of claim 7, wherein the plurality of sensors includes a button type sensor.
